(19) United States
(12) Patent Application Publication
(10) Pub. No.: US 2016/0338646 A1
(43) Pub. Date: Nov. 24, 2016

(54) ULTRA-THIN WEARABLE SENSING DEVICE

(71) Applicant: MC10, INC., Lexington, MA (US)

(72) Inventors: Stephen LEE, Ann Arbor, MI (US); Weihua LI, Acton, MA (US); Milan RAJ, Cambridge, MA (US); Roozbeh GHAFFARI, Cambridge, MA (US); Gilbert Lee HUPPERT, Stoneham, MA (US); Nicholas KUBASTI, Seattle, WA (US); Adam LEECH, Newton, MA (US); Hoi-Cheong Steve SUN, Lexington, MA (US); Scott SHAW, Waltham, MA (US)

(21) Appl. No.: 15/160,631

(22) Filed: May 20, 2016

Related U.S. Application Data
(60) Provisional application No. 62/164,301, filed on May 20, 2015.

(51) Int. Cl. A61B 5/00; A61B 5/0408; A61B 5/145; A61B 5/04

(52) U.S. Cl. CPC A61B 5/6832; A61B 5/04004; A61B 5/0059; A61B 5/04085; A61B 5/0022; A61B 5/4875; A61B 5/4266; A61B 5/14542; A61B 2560/0214; A61B 2562/0271; A61B 2562/0219; A61B 2562/166; A61B 2562/14

(57) ABSTRACT

An ultra-thin wearable sensing device includes a sensor tag IC that enables the device to communicate wirelessly to a reading device. The wearable sensing device includes one or more sensors connected to the sensor tag IC that sense characteristics of the person, animal or object that the sensing device comes in contact with. The sensed characteristics can include biological signals (e.g., ECG, EMG, and EEG), temperature, galvanic skin response (GSR), heat flux and chemicals or fluids released by the skin. The reading device can display the information to the user and/or transmit the sensor data to a remote location for further processing. A doctor can review the data or have the data further analyzed and use this data or information to assist with treatment.

### CROSS-REFERENCE TO RELATED APPLICATIONS

This application claims any and all benefits as provided by law including benefit under 35 U.S.C. §119(e) of the U.S. Provisional Application No. 62/164,301, filed May 20, 2015, the contents of which are incorporated herein by reference in its entirety.

### STATEMENT REGARDING FEDERALLY SPONSORED RESEARCH

Not Applicable.

### REFERENCE TO MICROFICHE APPENDIX

Not Applicable

### BACKGROUND

1. Technical Field of the Invention

The present invention is directed to wearable sensing devices that can be adhered to or worn on the body. More specifically, the invention is directed to wearable sensing devices having an ultra-thin or tattoo-like form factor and can be used to measure heart rate, temperature and other biometric information.

2. Description of the Prior Art

Some patients may suffer from intermittent atrial fibrillation. The intermittent nature of the abnormal heart beat makes it difficult for a doctor to observe the condition and the heartbeat wave form to provide treatment. In addition, the care providers (e.g., the hospital and the electrophysiologist) want to better monitor and bring patients into the clinic earlier since their reimbursements and payment methods have changed from pay for procedure to pay for outcome, and care providers are better able to achieve better outcomes if they can catch symptoms faster.

### SUMMARY

Therefore, there is a need for a device that enables the care providers to monitor their patients on a continuous basis and on an as needed basis, such as through the use of a portable, body worn sensor device. The devices and systems according to the present invention provide an ultra-thin wearable sensor device that can be used to monitor vital signs, such as heart beat, respiration and temperature of a patient. The wearable device according to the invention can operate by drawing power from an external source, such as an RFID or NFC reader. Alternatively, the wearable device can include an on-board power source, such as a battery or capacitor.

In accordance with some embodiments of the invention, a wearable device can include one or more sensors that can be used to measure biological signals of biological conditions such as ECG, temperature, GSR, heat-flux and chemicals/fluids using harvested power from an Near Field Communications (NFC)-enabled phone for a brief measurement (fractions of a second to as long as user holds phone up to device). The biological signals produced by the sensors can be filtered and analyzed by the microcontroller on the wearable sensor, the sensor reading device (e.g., smartphone) and by the cloud service where the data is ultimately sent. In addition, the wearable device can be used with a service—such as detection of arrhythmia. Other services may be include a call automatically made to your doctor; messages sent to loved ones; ambient conditions (lights) of the room change etc.

In accordance with some embodiments of the invention, the wearable device can be configured to measure and report sensed conditions for a predefined period of time or continuously for as long as it is able to harvest power from the reader. In accordance with some embodiments of the invention, the wearable sensor device can be used with loop-monitors and Holter monitors. A Holter monitor can measure and record ECG signal continuously for 24 hours or more. A loop monitor can measure and record ECG signals continuously for a week or month. The use of either of those devices may require a data service to scan through the ECG waveform data to find the atrial fibrillation or other abnormal heartbeat signal.

In accordance with some embodiments of the invention, the wearable device can include power source (such as an ultra-thin battery) to enable the device to measure and log sensed conditions continuously. The sampling rate of the wearable device can be adjustable, enabling the wearable sensor to measure and log heart-rate and other sensor data for hours, days and weeks or more. In some embodiments of the invention, the wearable sensor can measure biometric data continuously and search for signatures in the input data. A portion of the input data that includes the signature can be stored for later retrieval or transmission to a remote system for further analysis. For example, the wearable sensor can monitor ECG and when it detects atrial fibrillation, it will log that segment of data onto memory for later retrieval and transmission to a remote system (e.g., a cloud based system) for analysis.

In accordance with some embodiments of the invention, the wearable sensor device can be worn like a tattoo or a sticker that can be placed on the user's chest when the user detects an irregular heart rate. The user or a care giver can then place a sensor reading device (e.g., an NFC-enabled or RFID enabled smart phone or NFC/RFID reader) near the wearable sensor device to power the device and begin acquiring ECG, respiration and/or temperature data. That data can be stored and viewed on the reading device. In addition, where the reading device is connected to a network, such as the internet, the data collected can also be sent to the cloud for storage and further analysis. Additional services can also be provided for the data stored in the cloud. In accordance with some embodiments, a scanning service can analyze the heart beat data, the respiration data and/or the temperature data to identify irregular patterns in the individual signals (e.g., heartbeat signal data, respiration signal data and temperature signal data). Care givers can review the collected ECG waveforms remotely and then communicate (e.g., by voice, email or text message) with patients exhibiting pathologies to arrange for a checkup with an appropriate care provider (e.g., a doctor or cardiac specialist).

In accordance with some embodiments of the invention, the wearable sensor device can be a completely passive device that receives power from the NFC-enabled handset and can be reusable or disposable after one or more uses.

These and other capabilities of the invention, along with the invention itself, will be more fully understood after a review of the following figures, detailed description, and claims.

### BRIEF DESCRIPTION OF THE FIGURES

The accompanying drawings, which are incorporated into this specification, illustrate one or more exemplary embodiments of the inventions and, together with the detailed description, serve to explain the principles and applications of these inventions. The drawings and detailed description are illustrative, and are intended to facilitate an understanding of the inventions and their application without limiting the scope of the invention. The illustrative embodiments can be modified and adapted without departing from the spirit and scope of the inventions.

FIG. 1 shows the sensor tag IC (AMS SL13a) handles NFC communication, energy harvesting and analog to digital conversion, sensor signal processor IC (AD8232) serves as the heart rate monitor—rejecting common-mode signals, and performing high and low pass filtering and the op-amp (ADA4051-1) performs a level-shift.

FIGS. 2A-2D show plots of the S parameters of a sensor device antenna design having a 12 mil trace, 5 mil space and 3 turns.

FIGS. 3A-3C show various sensor device configurations according to some embodiments of the invention p FIG. 4 shows a diagrammatic view of a sensing device (297-00070 configuration) configured with a 25 mil trace, 5 mil space and 4 turns.

FIG. 5 shows a diagrammatic view of a sensing device (297-00071 configuration) configured with a 12 mil trace, 5 mil space and 3 turns.

FIG. 6 shows a diagrammatic view of a sensing device (297-00072 configuration) configured with a 5 mil trace, 5 mil space and 6 turns.

FIG. 7 shows a diagrammatic view of a sensing device (297-00073 Pempamsie configuration) configured with a 20 mil trace, 15 mil space and 6 turns.

FIG. 8 shows a Smith plot of the device of FIG. 4 (297-00070 configuration) touching the skin (palms).

FIG. 9 shows a system for reading heart rate, temperature and heart wave according to some embodiments of the invention;

FIG. 10 shows the system of FIG. 9 reading sensor signals through the patient's clothing.

FIG. 11 shows a Pempamsie (50×90 mm) antenna configuration of a sensor device according to some embodiments of the invention attached to the forearm to test the antenna coil against skin.

FIG. 12 shows a diagrammatic view of a sensing device according to some embodiments of the invention.

FIG. 13 shows a diagrammatic view of a sensing device according to some embodiments of the invention.

FIG. 14 shows a diagrammatic view of a sensing device according to some embodiments of the invention.

FIG. 15 shows a diagrammatic view of a sensing device according to some embodiments of the invention.

FIG. 16 shows a diagrammatic view of a sensing device according to some embodiments of the invention.

FIG. 17 shows a diagrammatic view of a sensing device according to some embodiments of the invention.

FIG. 18 shows a diagrammatic view of a sensing device according to some embodiments of the invention.

FIG. 19 shows a diagrammatic exploded view of a sensing device according to some embodiments of the invention.

FIGS. 20 and 21 show a bottom view of a sensing device according to some embodiments of the invention.

FIG. 22 shows a diagrammatic view of the stack-up construction of a sensing device according to some embodiments of the invention.

### DETAILED DESCRIPTION OF PREFERRED EMBODIMENTS

The present invention is directed to a wearable sensor device that can be worn like a tattoo or a sticker and can be placed on the user's body (e.g., chest) when the user detects a health concern or wants to monitor biometric information for a period of time. The user or a care giver can then place a sensor reading device (e.g., a smart phone or NFC/RFID reader) near the wearable sensor device to power the device and begin acquiring ECG, respiration and/or temperature data. The measured biometric data can be stored on the sensor reading device and transferred over a network (e.g., the Internet) to a remote storage location (e.g., cloud storage) where the data can be reviewed by a care giver or analyzed to detect conditions associated with a health risk.

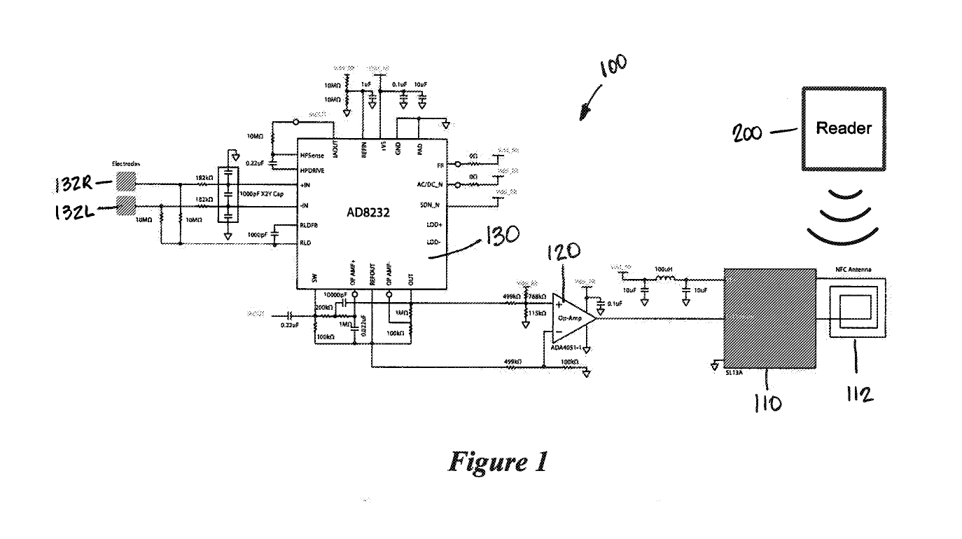

An example of a system 100 according to some embodiments of the invention is illustrated in FIG. 1. In accordance with some embodiments, a balance can be made between conformability, size, functionality and cost. In accordance with some embodiments of the invention, the sensing device can include a sensor tag integrated circuit (IC) 110 (e.g., an SL13a from ams AG, Unterpremstaetten, Austria), an operation amplifier 120 (e.g., an ADA4051-1 from Analog Devices, Inc., Norwood, Mass.) and a sensor signal processor circuit 130 (e.g., an AD8232 from Analog Devices, Inc., Norwood, Mass.). Additional passive components can be used to provide signal tuning. The sensor tag IC 110 can include a sensor input that can be connected to an external sensor (such as through the sensor signal processor 130) to receive sensor data (e.g., biological signals or features) and transmit the sensor data to a sensor reading device 200. An operation amplifier 120 can be provided to level shift, and increase the signal levels for interfacing with the sensor tag IC 110. In accordance with some embodiments, the amplification can be provided by components within the sensor tag IC.

In the illustrative embodiment, the sensor tag IC 110 provides energy harvesting from the Near Field Communication (NFC) reader 200 provides analog to digital conversion of the heart beat and temperature signals from the sensor (or sensor signal processor IC) and provides the NFC communication with the sensor reading device 200. The sensor tag IC 110 can provide electricity to power the sensor signal processor 130 and any other components of the system 100. The sensor signal processor IC 130 can be connected to electrodes 132R, 1323L and can serve as a heart rate monitor—rejecting common-mode signals, and performing high and low pass filtering. Where necessary, an amplifier 120 can be included to perform a signal level-shift.

While the illustrative example describes the invention in the context of a wearable sensing device that senses heart beat signals and temperature, any other sensing modalities, such as motion (e.g., using an accelerometer and/or gyroscope), sound and vibration (e.g., using an accelerometer and/or a microphone), hydration and perspiration (e.g., using electrodes, galvanic skin response, capacitive sensors) and light (e.g., using photodiodes and optical transducers) can be used. The only limitation is the ability of the wearable sensor to harvest power from the sensor reading device or to be powered by an attached power source. Other signals that can be measured include bio-potentials (e.g., ECG, EMG, and EEG) and bio-impedances, galvanic skin response (GSR), temperature, heat flux, blood oxygen, and chemical or fluid analysis. The signals can be filtered and/or analyzed by a processor in the wearable sensing device, on the sensor reading device, and/or a remote storage location (e.g., cloud storage, or a server managed by a care provider).

In FIG. 1, starting from the left side of the diagram, the electrodes can have series resistors that serve as current limiters, protection against static discharge and RFI filtering in conjunction with the X2Y capacitor. The X2Y has matched capacitors which help maintain high common-mode rejection. The heart-rate sensor monitor IC 130, can have two gain stages. The first stage can be set to about 100 so the majority of gain can be in-front to maximize the signal to noise ratio. The first stage gain can be set higher (e.g. 1000 or more) or lower (e.g., 10 or less) depending on the signal from the electrodes. In other words, the referred to input noise should be low by establishing the majority of gain up front. The sensor monitor IC 130 includes an adjustable high-pass filter that can be configured with a cut-off at about 7 Hz. Note since the gain is 100, the 7 Hz poles effectively attenuate 40 dB at 0.007 Hz. Right leg drive circuitry can be utilized, albeit by driving the input pins via the 10 MΩ resistors. The signal from the first stage is then passed to the second stage which can be set at a gain of about 11. The second stage gain can be set higher (e.g. 100 or more) or lower (e.g., 2 or less) depending the input signal and requirements of the sensor tag IC 130 input. A second high-pass pole, via an ac-coupling network between the first stage and the second gain stage at 7 Hz can be provided as well. The second stage gain can be configured in a two-pole low-pass Sallen-Key filter configuration.

In accordance with some embodiments of the invention, the operation amplifier (e.g., the ADA4051-1) can be configured as a difference amplifier that operates as the level-shifter to shift the voltage output from the AD8232 to conform to the 0.3V-0.6V input range of the sensor tag IC (e.g., the SL13a).

The sensor tag IC 110 can be configured to harvest power from the sensor reading device (e.g., a handset or smartphone) and provide electric power to each of the components of the sensor device system 100. In accordance with some embodiments, the power signal that is harvested is noisy and performance can be improved by filtering the power signal. In accordance with some embodiments, a pi filter consisting of two 10 uF capacitors and a 100 uH inductor can be used to smooth out the noise and spurs from the power signal before delivering the power to the sensor signal processor IC 130 and the amplifier 120.

In accordance with some embodiments of the invention, any sensor tag IC 110 that can effectively harvest enough energy to power the system 100 can be used. In accordance with some embodiments, a sensor tag IC 110 that can provide sensing via an analog to digital converter (ADC) or via serial digital input and can energy harvest (e.g., RF or NFC), can be used. Other NFC enabled processors that can be used include the RF430FRL15xh, (Texas Instruments, Inc., Dallas Tex.), and the NXP NHS3100, and the NXP Melexis 90129 (NXP Semiconductor N V, Eindhoven, the Netherlands).

In accordance with some embodiments, any sensor signal processor IC can be used depending on the sensor used and signal levels produced. In the illustrative embodiment, the Analog Devices AD8232 sensor signal processor IC 130 is a low-power, low-cost, heart rate monitor IC. The AD8232 provides an analog output, common-mode rejection, common-mode bias servoing (via right leg drive which can be driven through the Left/Right electrodes), fast recovery to saturated inputs, high-pass and low-pass filtering on a single chip (IC). In accordance with some embodiments, the sensor signal processor IC can provide a digital output which can communicate directly to a digital input of the sensor tag IC.

In accordance with some embodiments of the invention, the amplifier 120, ADA4051-1, can be an ultra-low power, zero-drift op amp. While the system does not require a zero-drift amp, the ADA4051 has very low 1/f noise because of the auto-zero and chopping. In some embodiments, depending on the input requirements of the analog to digital converter (ADC) of the sensor tag IC 110, level shifting (and the associated amplifier components) are not needed.

In accordance with some embodiments of the invention, a pi filter with poles set at frequency that can filter a 1 μs edge-to-edge of a 9 μs pulse for a duration of 5 ms can be used. Preferably, the Pi filter includes 10 μF or 15 μF capacitors and a 100 μH inductor. Alternatively, the Pi filter can include 15 μF capacitors and a 100 μH inductor which provides a 3 pole filter at ˜4k Hz which is lower than the noise spurs generated by the SL13a's harvested power line.

In accordance with some embodiments, the wearable sensing device can be configured to operate without a battery and receive power by harvesting energy from the wireless or radio frequency (RF) signals provided by the sensor reading device (e.g., NFC signals). In accordance with some embodiments of the invention, the wearable sensor device can be in close proximity to the sensor reading device to enable continuous operation for extended periods of time (e.g., 1-60 seconds, 1-60 minutes, 1-24 hours, and from 1 day to a year or more). In accordance with some embodiments of the invention, the wearable sensor device can include a battery (or other power source, such as, a capacitor or solar cell) that powers the device on a continuous basis. A sensor reading device 200 can be used intermittently to collect the data and optionally, recharge the battery. Alternatively, the battery powered sensor reading device can wirelessly (e.g., Bluetooth, WiFi, ZigBee, IR) or by wire (e.g., USB, Ethernet, SPI) connect to an interface that collects the data in near real time.

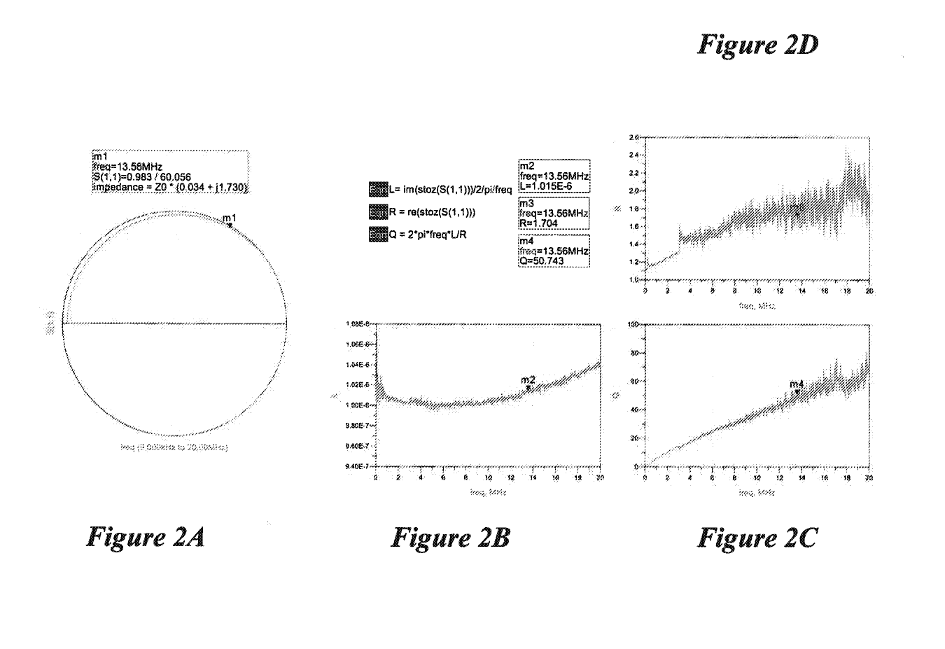

In accordance with some embodiments, the antenna can be designed to surround the electrodes and circuitry. This configuration can be used to maximize the area inside the antenna coil to more closely match the antenna on the handset which can be used to improve energy harvesting. FIGS. 2A-2D show some plots of the S parameters of the antenna design according to some embodiments of the invention. FIG. 2A shows a Smith plot of an antenna according to some embodiments of the invention. FIGS. 2B and 2D show plots of the imaginary and real components of the antenna impedance with frequency. FIG. 2C shows a plot of the Q factor of the antenna with frequency.

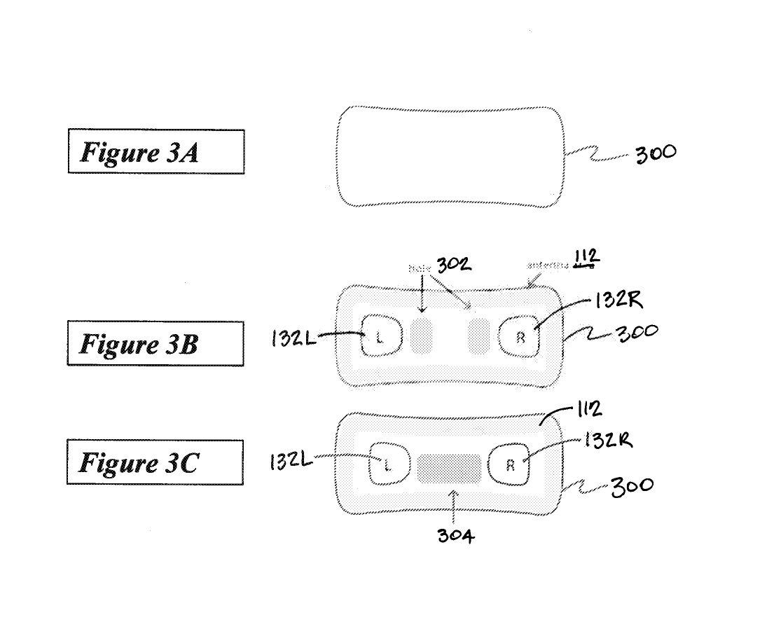

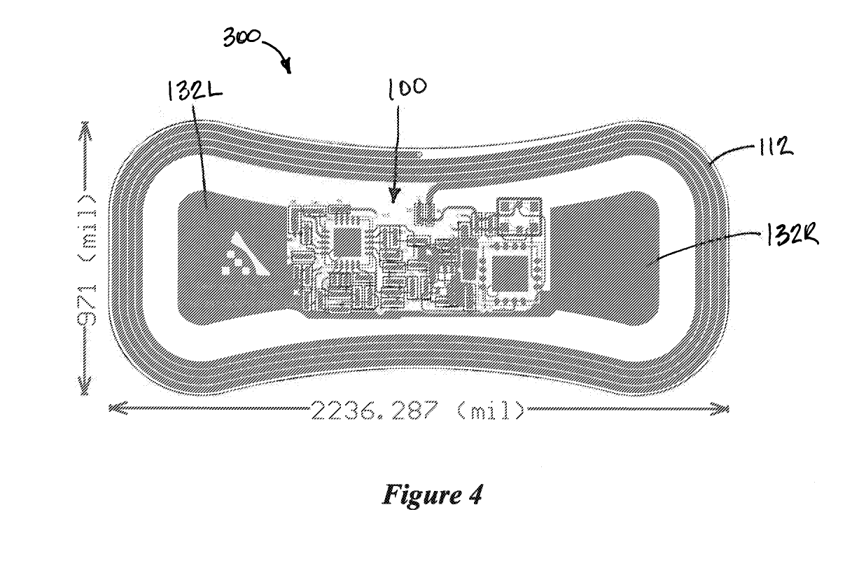

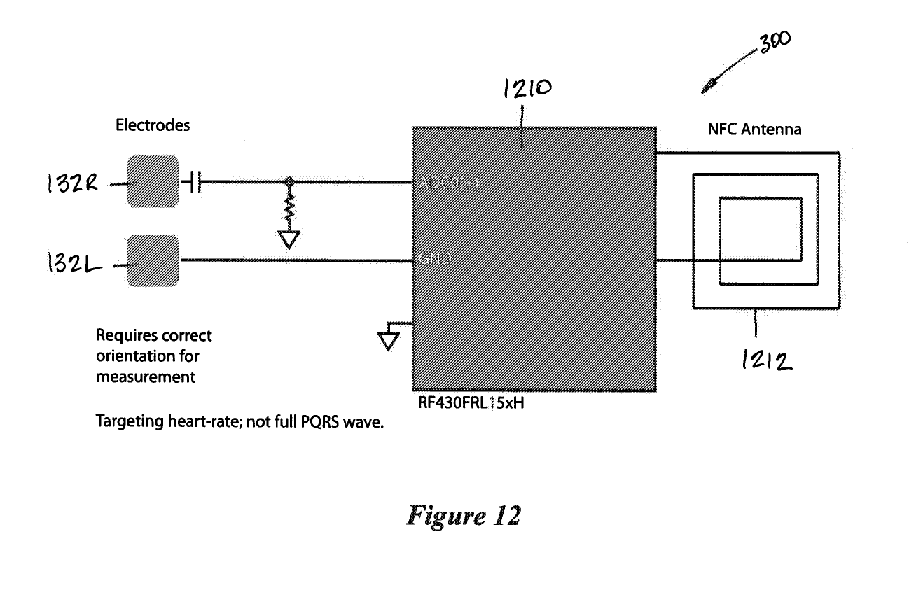

In accordance with some embodiments of the invention, the shape of the antenna can be selected to provide optimum power harvesting while producing a relatively small size sensing device 300, as shown in FIGS. 3A-3C. The shape of the sensing device 300 can be a function of a) the size of the antenna 112; and b) the spacing and size of the electrodes. In accordance with some embodiments, the size of the device 300 can be approximately 25 mm×55 mm and be configured in a “bowtie” configuration as shown in FIGS. 3A-3C. In this configuration, the antenna 112 can extend around the periphery of the sensor device and the components, including the integrated circuits and electrodes 132R, 132L can be placed in the open central region of the device, inside the antenna 112. The sensing device 300 can include holes or opening 302, 304 that can be provided to accommodate the electronic circuits, such as those shown in FIGS. 12-18.

In accordance with some embodiments of the invention, the sensing device 300 can include a pressure sensitive adhesive (e.g., an acrylic adhesive) that can be used to adhere the device 300 to the body. The bottom surface of the sensing device 300 can include electrodes 132R and 132L that are spaced apart on the device. The pressure sensitive adhesive can include openings that enable the electrodes 132R, 132L to directly contact the skin of the user. The surface finish of the electrodes 132R, 132L can be configured (with or without an intervening conductive medium) to directly contact the patient's skin. In accordance with some embodiments of the invention, the surface finish of the electrodes 132R, 132L on the bottom side of the flexible printed circuit board (FPCB) can be electroless nickel immersion gold (ENIG) or immersion silver. Optionally, a layer of silver-silver-chloride ink can be applied (e.g., printed) over the surface finish.

FIGS. 4-7 shows diagrammatic views of various antenna configurations according to various embodiments of sensing devices 300 according to the invention. Each sensing device 300 can include an antenna 112 arranged around the peripheral edge of the device 300 leaving the central portion of the device open for the system circuit 100 and the electrodes 132R, 132L.

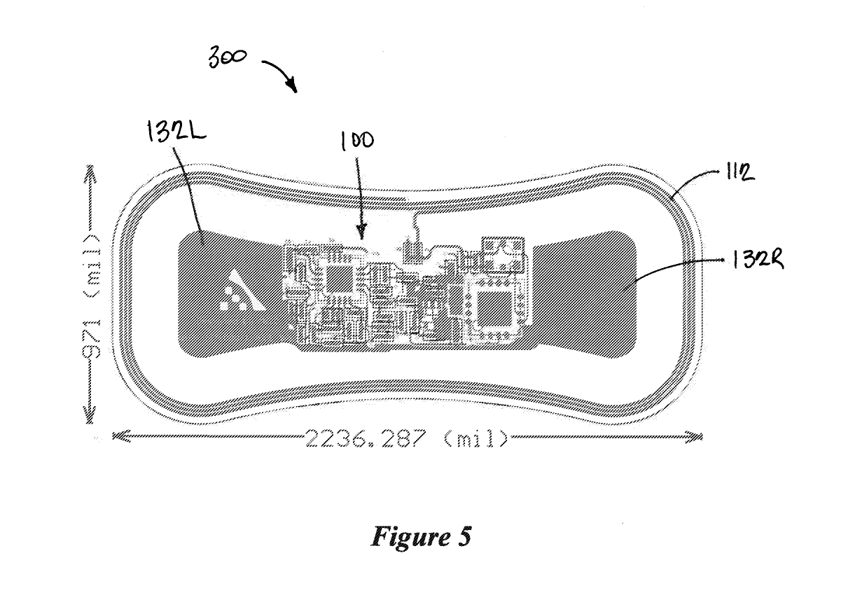

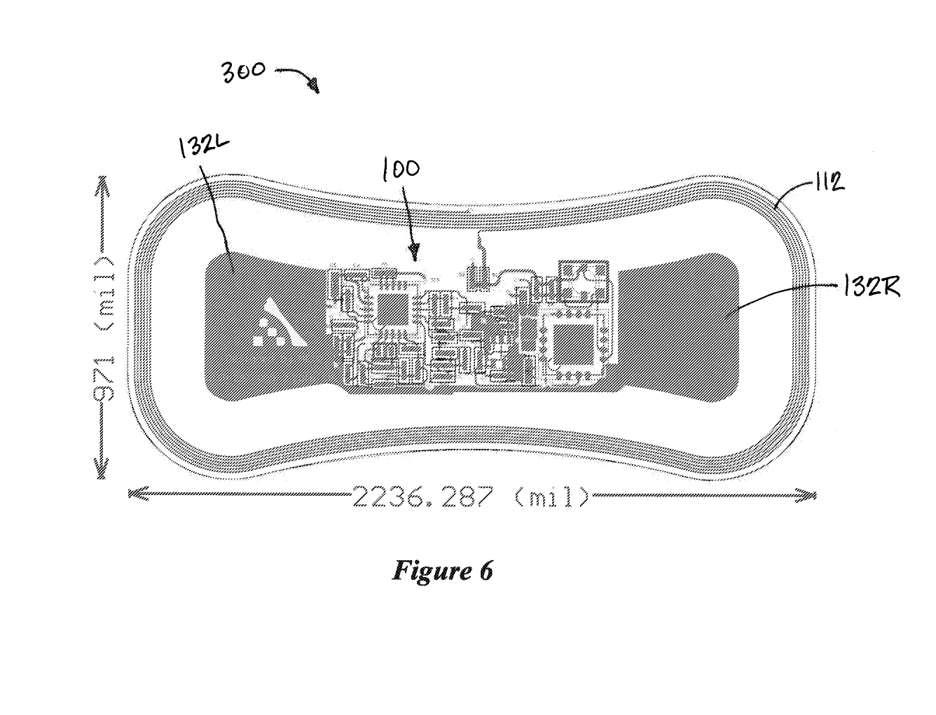

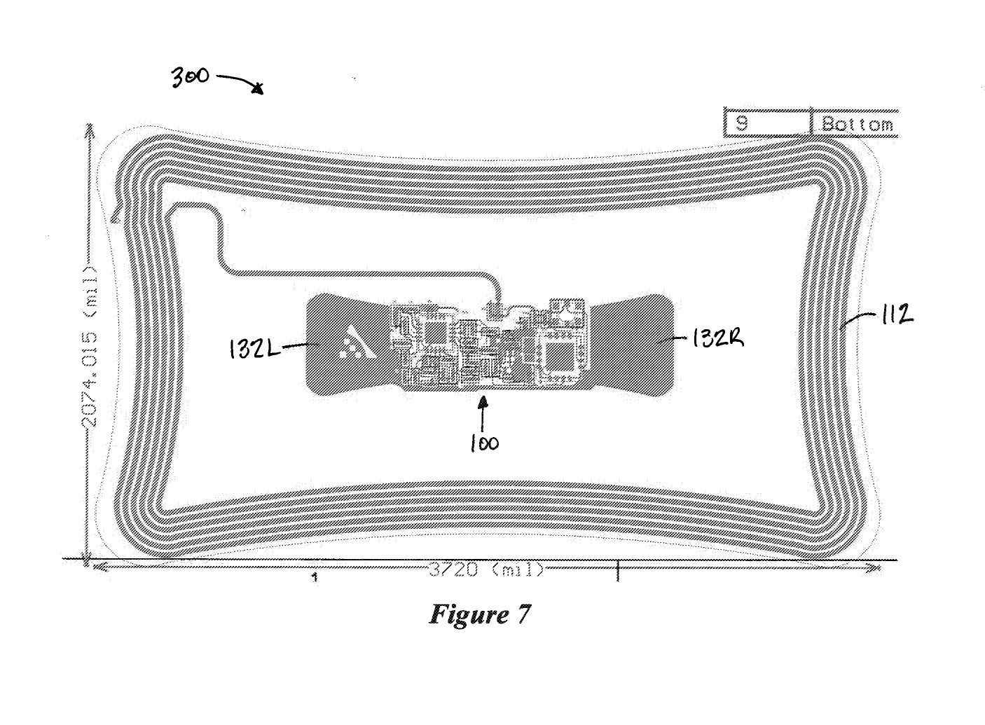

FIG. 4 shows sensor device 300 according to some embodiments of the invention having an antenna 112 constructed with a 25 mil trace, a 5 mil space and 4 turns. FIG. 5 shows sensor device 300 according to some embodiments of the invention having an antenna 112 constructed with a 12 mil trace, a 5 mil space and 3turns. FIG. 6 shows sensor device 300 according to some embodiments of the invention having an antenna 112 constructed with a 5 mil trace, a 5 mil space and 6 turns. FIG. 7 shows sensor device 300 according to some embodiments of the invention having an antenna 112 in a Pempamsie configuration constructed with a 24 mil trace, a 15 mil space and 6 turns.

A coarse capacitor and fine capacitor can be used to tune each antenna 112. The thickness of the metal traces that form each antenna 112 can affect the tuning values. For instance, the device shown in FIG. 5 (12 mil trace, 5 mil width, 3 turn design) was fabricated using a standard 0.062 inch rigid FR4 process on 1 oz copper. The tuning capacitor values for the device shown in FIG. 5 were different from same design fabricated using a 200 um flex circuit on ¼ oz. copper. Furthermore, even on flex, the tuning capacitors may vary. In addition, the tuning capacitor values can differ depending on other fabrication process and materials, such as the solder mask used or whether an organic solderability preservative (OSP) was used.

In accordance with some embodiments of the invention, a layer of thin foam can be applied to the top of the electronics 100 to increase resistance to electro-static discharge that can damage the circuit components.

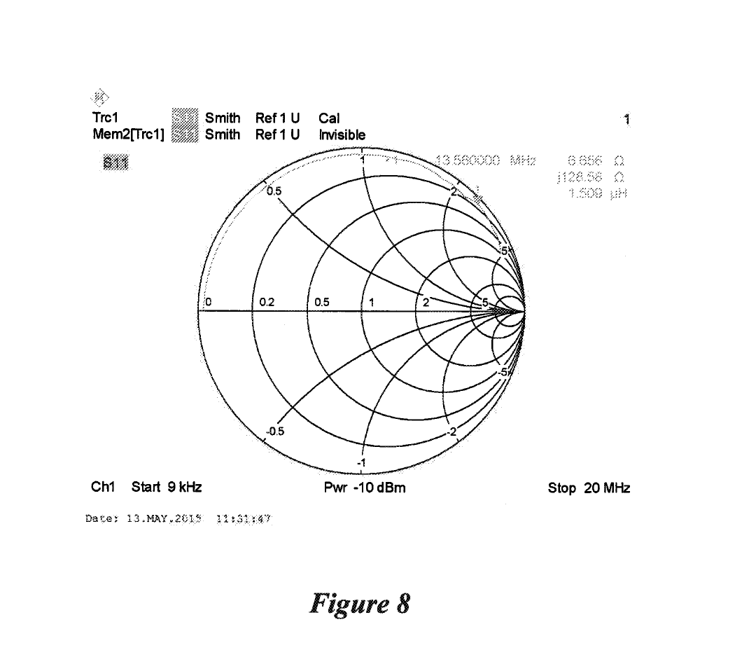

Tuning can be performed in many configurations, however one preferred configuration is to use a configuration that is similar to the configuration of the intended use, for example, against the skin. For evaluation purposes, three configurations were tested: 1) device floating in air; 2) with thumbs pinching electrodes and part of antenna; 3) with device on the palm of the hand. The Smith chart shown in FIG. 8 shows the difference in the various configurations. Table 1 shows the variation in fabrication processing.

TABLE 1
Variation in coarse and fine measurement capacitors

| Design, Cu Weight, Fab | Ccoarse | Cfine |
|---|---|---|
| 12 mil trace, 5 mil space, 3 turn on 1 oz Cu FR4 | 100 pF | 12 pF |
| 25 mil trace, 5 mil space, 4 turn on ½ oz. Flex | 68 pF | 8.2 pF |
| (Gold Phoenix STLE09CA022886) | | |
| 25 mil trace, 5 mil space, 4 turn on ½ oz. Flex | 75 pF | None |
| (Gold Phoenix SA-0158 ILT1504-077) | | |

Table 2 shows the different computed tuning capacitance values for the various designs in various configurations. The design shown in FIG. 4 has a 25 mil trace, 5 mil space and 4 turns. The design shown in FIG. 5 has a 12 mil trace, 5 mil space and 3 turns. The design shown in FIG. 6 has a 5 mil trace, 5 mil space and 6 turns. The design shown in FIG. 7 has a 24 mil trace, 15 mil space and 6 turns.

TABLE 2
Computed tuning capacitances for the 4 embodiments

| | | | calculated | |
|---|---|---|---|---|
| | | measured | capacitance | |
| | | inductance | in pF | Calculated |
| model | surface | (μH) | (C_{tuning} + C_{SL13A}) | C_{tuning} in pF |
| 297-00070 | air | 1.327 | 103.8125745 | 78.81257447 |
| 297-00070 | table | 1.407 | 97.90994053 | 72.90994053 |
| 297-00070 | skin (palm) | 1.509 | 91.29177358 | 66.29177358 |
| 297-00070 | thumbs | 1.416 | 97.28763159 | 72.28763159 |
| 297-00071 | air | 1.006 | 136.9376604 | 111.9376604 |
| 297-00071 | table | 1.015 | 135.7234348 | 110.7234348 |
| 297-00071 | skin (palm) | 1.077 | 127.9102009 | 102.9102009 |
| 297-00071 | thumbs | 1.018 | 135.323464 | 110.323464 |
| 297-00072 | air | 4.064 | 33.89746219 | 8.897462187 |
| 297-00072 | table | 4.734 | 29.09997599 | 4.099975988 |
| 297-00072 | skin (palm) | 4.714 | 29.22343791 | 4.223437914 |
| 297-00072 | thumbs | 4.404 | 31.2804919 | 6.2804919 |
| 297-00073 | air | 6.619 | 20.81270378 | −4.187296219 |
| 297-00073 | table | 9.684 | 14.22545295 | −10.77454705 |
| 297-00073 | skin (palm) | 9.419 | 14.62568068 | −10.37431932 |
| 297-00073 | thumbs | 6.969 | 19.76743956 | −5.232560435 |

In operation, the sensor tag IC 110 can include a state machine that can be configured to measure a set of multiplexed external signals, such as an ECG signal and an internal temperature sensor, using an internal analog to digital converter (ADC). A computer program, such as a sensing application executed on the sensor reading device 200 (e.g., an NFC-enabled handset) can be used to control the sensing process. In accordance with some embodiments of the invention, in operation, the sensor reading device 200 can be positioned close to the sensing device 300 worn on the patient's body, causing the sensing device 300 to power it up by harvesting energy from NFC RF field generated by the sensor reading device. Next, the sensing application is started and the sensing device 300 can be initialized to get it ready for sensing and communication with the sensor reading device 200 over the NFC interface. The sensing device begins continuous real-time sensing and monitoring of skin temperature and ECG signal from the patient:
a) Start the ECG sensing and poll the sensing device 300 for data by sending a command over NFC interface to the sensing device 300. The polling process can start with the initiation of an ECG signal measurement and end when the measurement is completed the result is returned to the sensor reading device 200.

b) Save the data stream locally to a file on the sensor reading device 200 and also plot the live ECG data on sensor reading device display.

c) Feed the data stream to heart-rate algorithm (e.g., developed based on a QRS detection algorithm) to calculate real-time heart rate and update on the sensor reading device display.

d) Switch channels to sense skin temperature and update temperature on the sensor reading device display.

Go back to step (a) and continue until the process is stopped, such as, by the user.

After the conclusion of the measurement, the user has the option to either forward the data generated to physicians via email or push it to a cloud based storage system for further review and analysis.

One of the advantages of the system 100 according to the invention, is that the wearable sensor can measure an instantaneous single-point condition such as temperature, as well as measure over several an extended period of time (e.g. from a few seconds to several minutes or hours). In accordance with some embodiments, the wearable sensor 300 can be configured to harvest power continuously for as long as the user has the sensor reading device 200 (e.g., smartphone) in close proximity to the wearable sensing device 300. This enables the wearable device 300 to produce hundreds or thousands of data points. This also enables the wearable device 300 to power many sophisticated sensors over long periods of time and enables many uses.

One application of invention is for upstream detection of irregular arrhythmia or intermittent atrial fibrillation. In some cases, arrhythmia is inconsistent and the patient may complain of palpitations but when visiting the clinic it cannot be observed. Furthermore, the use of a 1-week Holter monitor may not detect it either. A device according to the present invention can be offered in a box as a kit, akin to a box of adhesive bandages that can be stored in the patient's medicine cabinet. The patient applies the patch to her or himself upon onset of palpitations. The wearable sensing device provides a low cost service for detecting and capturing the palpation upon onset and having a clinician or algorithm review the heart wave to confirm and aid in determining treatment. The patient places a sensor reading device (e.g., a smartphone) near the wearable sensing device 300 and begins recording heart signal data. The sensor reading device 300 can be embedded in a mattress and/or a blanket to provide continuous monitoring and recording while the patient sleeps. The heart signal data can also be sent (e.g., wirelessly or by wire) to a remote storage location (e.g., cloud storage) for storage and analysis. The clinician can then call the patient in and offer treatment. Furthermore, signal data can be used by other services such as automatic calling to the clinician, loved ones, or adjusting parameters in the smart home such as heating, air-conditioning, lighting, or etc.

Another application for the invention is for heart rate monitoring. Heart failure is a common condition that affects nearly 6 million Americans. By 2030, the prevalence of heart failure will increase 46 percent from 2012 estimates. Corlanor® (ivabradine) is an oral medication indicated to reduce the risk of hospitalization for worsening heart failure in patients with stable, symptomatic chronic heart failure with left ventricular ejection fraction (LVEF) ≦35 percent, who are in sinus rhythm with resting heart rate ≧70 beats per minute (bpm) and either are on maximally tolerated doses of beta blockers or have a contraindication to beta blocker use. It is thus critical to monitor fluctuations in resting heart rate during daily intake of Corlanor. However, existing heart rate monitoring devices are expensive, bulky devices that require trained health care professionals to operate and instrument patients.

Embodiments according to the invention include a low-cost, disposable skin-worn ECG sensing device that enables rapid measurement of resting heart rate and skin temperature in the home environment. A patient can affix the wearable sensing device 300 to their chest at bed time (or any other time) and the device can monitor the heart beat signal. The wearable sensing device 300 can capture heart rate data and instantaneously transfer this data to a cloud server for data analysis, evaluation of compliance, and efficacy assessment (i.e. does resting heart rate trend above or below 70 bpm) over the course of medical treatment. The wearable sensing device 300 can be an ultra-thin and comfortable ECG sensor, which is used in conjunction with NFC-enabled readers and/or smart phones 200 (i.e. Android smart phones with NFC capability) for energy harvesting and data capture.

In accordance with some embodiments, the sensor reading device 200 (e.g., the NFC enabled handset) transmits its carrier signal via its antenna coil. Bringing the sensor reading device 200 in proximity to the wearable sensing device 300 couples the 13.56 MHz carrier from the transmitter coil on the sensor reading device with the coil 112 on the wearable sensing device 300

The sensor reading device 200 does not have to be a handset. It can be a door lock or a store counter. The transmitter can sense when the wearable sensing device 300 is in proximity and an app can be launched automatically by the operating system of the system that includes or is coupled to the sensor reading device 200. For instance, if the sensor reading device 200 recognizes a specific identifier, it can be used to authenticate a person and provide access to certain places or system features. For example, if the sensor reading device 200 is part of a car, it can be used to unlock the door or trunk of the car.

In addition, if the sensor reading device 200 recognizes certain unique biometric patterns, it can be used to open up other access points. For instance, it can be used to open a safe when it recognizes the user's sweat profile, iris pattern, blood etc.

In accordance with some embodiments, if the sensor reading device 200 is included in a smartphone, and includes an application that collects ECG data, the ECG data can be sent to a local or remote system or service, which analyzes and diagnoses arrhythmia. If it recognizes irregular heartbeats, it can inform the patient and the doctor. An appointment can be automatically setup or in worst-case, an ambulance can be sent.

In accordance with some embodiments, remote servers can log the data and monitor the biometrics and other environmental conditions. The services may act upon those conditions. For instance for home automation, if the wearable sensing device or the sensor reading device determines (e.g., by analyzing the received temperature data) that the wearer is cold, it can communicate with the home thermostat to increase the heat in a portion of the house (e.g., based on user profiles and/or GPS data). In health care settings, the information can be used to better refine drug therapy—by adjusting dosage from time to time.

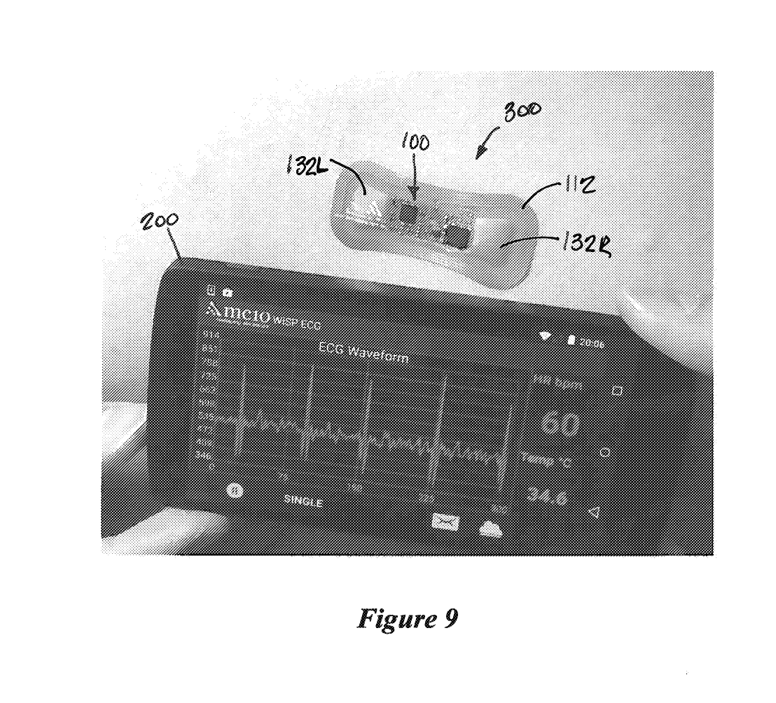

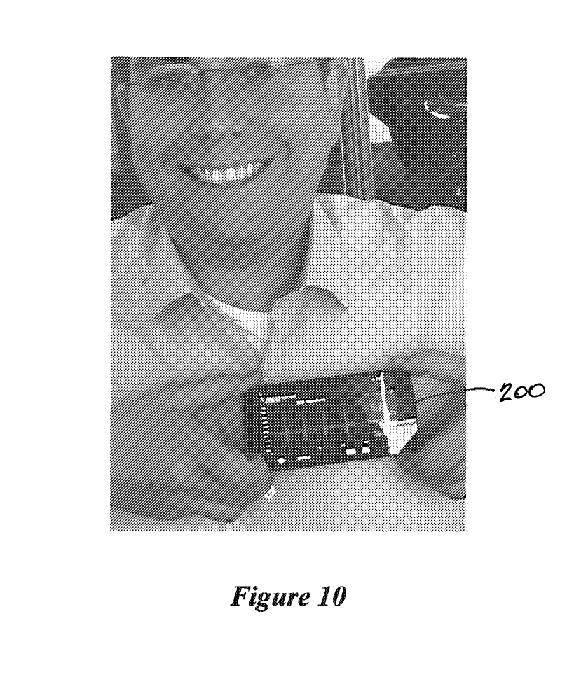

FIG. 9 provides an illustrative example of sensor reading device 200 according to some embodiments of the invention showing the ECG wave, the heart rate (HR) in beats per minute (bpm) and the skin temperature on the display, using data received from the sensing device 300 attached to the skin. FIG. 10 shows an illustrative example of the sensor reading device 200 according to some embodiments of the invention, receiving data from a sensing device through clothing of the patient.

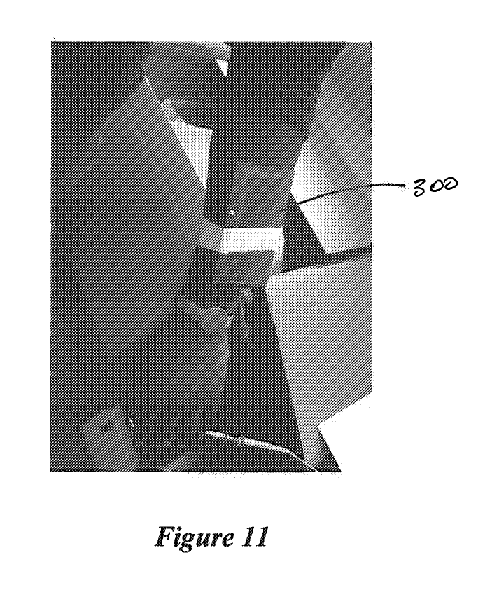

FIG. 11 shows an example of a system for testing how contact with skin impacts the ability of the NFC antenna 112 of the sensing device 300 to power the device 300 and transfer data.

FIGS. 12-17 show sensing devices 300 according to some embodiments of the invention. In these embodiments, additional circuitry can be included in the sensor tag IC to provide the functionality of the sensor signal processor circuit and/or the amplifier shown in FIG. 1.

FIG. 12 shows a sensing device according to some embodiments of the invention. In this embodiment, the sensor tag IC 1210 includes all the circuitry needed to receive the ECG signals from the electrodes 132R, 132L and communicate with the sensor reading device 200 using the NFC antenna 1212.

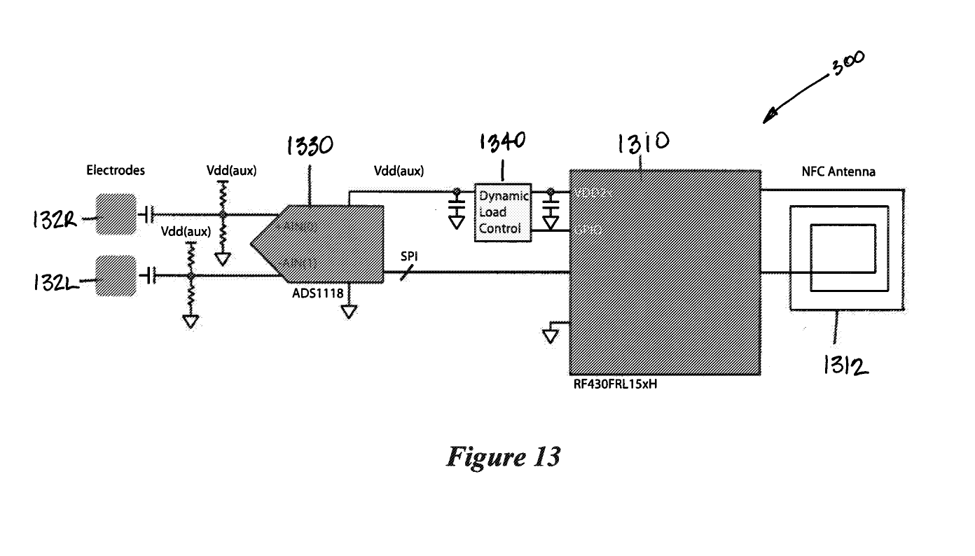

FIG. 13 shows a sensing device, similar to that of FIG. 12, according to some embodiments of the invention. In this embodiment, the sensing device includes a dynamic power sequencing circuit 1340 that enables the sensor tag IC 1310 more time to charge its internal storage capacitor before it powers on. The sensing device can also include an analog to digital converter and programmable amplifier IC (e.g., Texas Instruments (Dallas, Tex.) ADS1118) 1330 that can be used to amplify and convert analog sensor signals to digital values for storage in the sensor tag IC 1310 and for transmission to the sensor reading device 200.

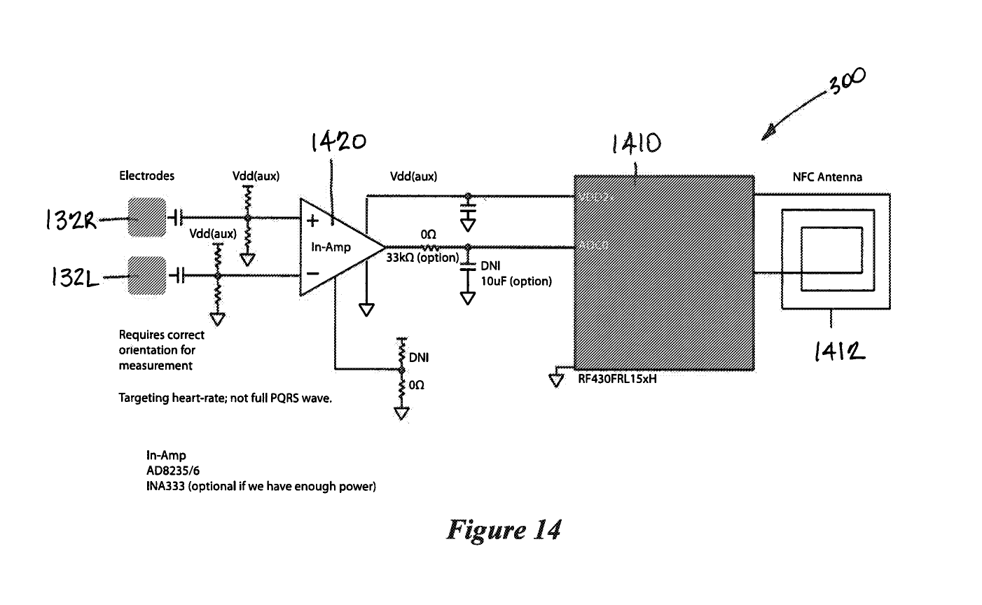

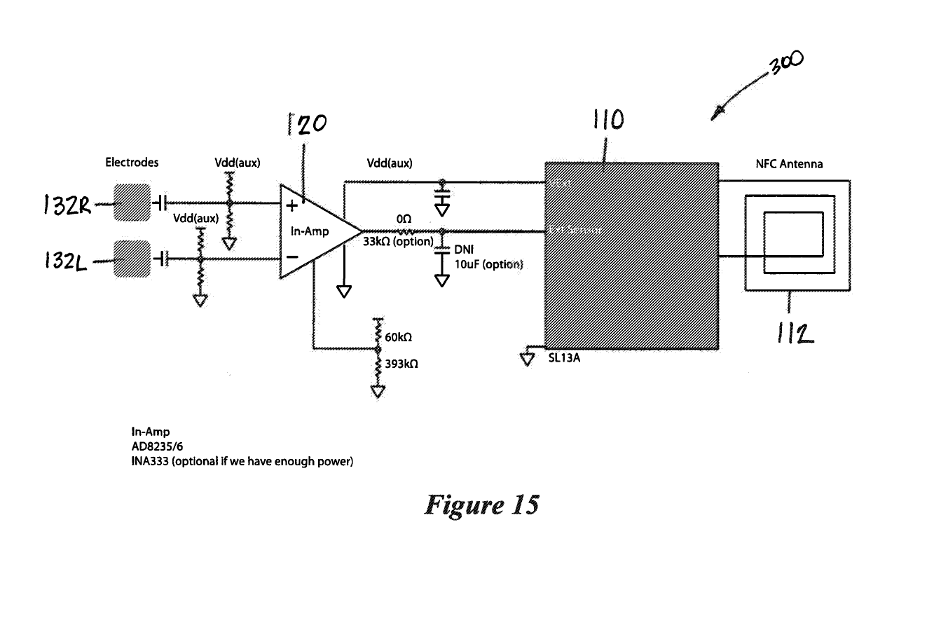

FIGS. 14 and 15 show a sensing device, similar to that of FIG. 12, according to some embodiments of the invention. In this embodiment, the sensing device includes a low power instrumentation amplifier 1420, 120 to amplify the ECG signals input to the sensor tag IC 1410, 110.

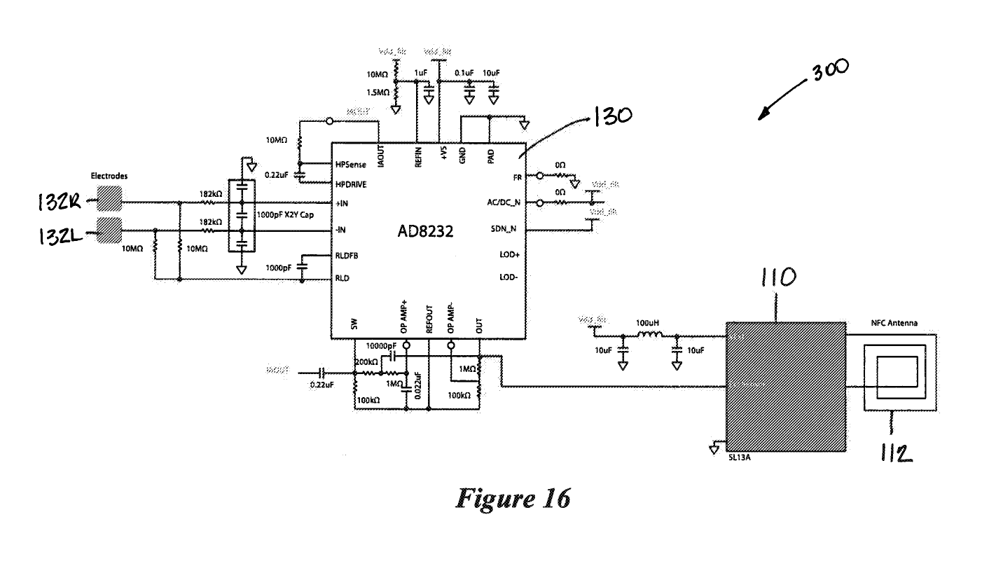

FIG. 16 shows a sensing device, similar to that of FIGS. 1 and 12, according to some embodiments of the invention. In this embodiment, the sensing device includes a sensor signal processor IC 130 that processes the ECG signals from the electrodes 132R, 132L before they are input into the sensor tag IC 110.

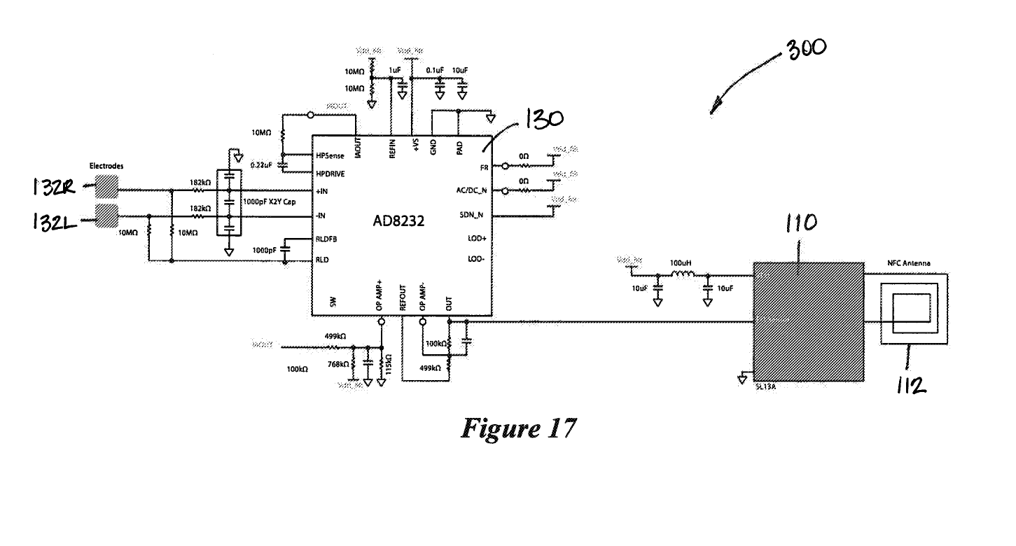

FIG. 17 shows a sensing device, similar to that of FIG. 16, according to some embodiments of the invention. In this embodiment, the low pass filtering is reduced from 2 poles to 1 pole and the output op-amp of the sensor signal processor IC 130 is adjusted to amplify the ECG signal sent to the sensor tag IC 110.

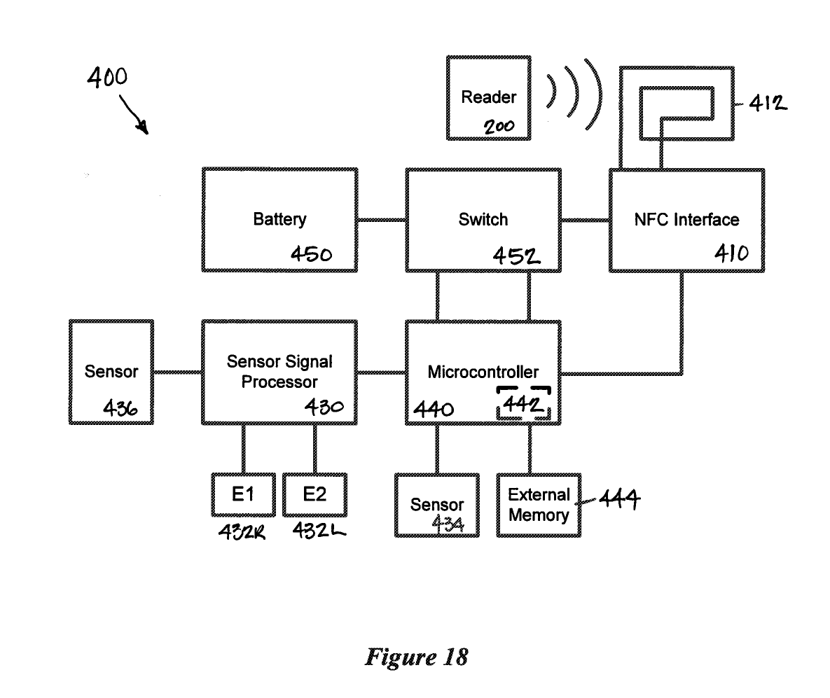

FIG. 18 shows a diagrammatic view of a sensing device 400 according to some embodiments of invention. The sensing device 400 includes an interface 410 (such as the NFC sensor tag IC 110, SL13A), connected to a microcontroller 440 and a switch 452. The sensing device 400 can also include a battery 450 connected through the switch 452 and one or more sensor signal processors 430 connected to the microcontroller 440. Electrodes 432R, 432L can be connected to the sensor signal processor 430. Other sensors, such as accelerometers, gyroscopes, temperature sensors, optical sensors, and sound/vibration sensors can be connected to the sensor signal processor 430 and/or directly to the microcontroller 440. The microcontroller 440 can include internal memory 442 and external memory 444 for storing programs and data.

The microcontroller 440 can be a low-powered, general purpose microcontroller, such as an MKL02Z32CAF4R microcontroller from Freescale Semiconductor (Austin, Tex.), now part of NXP Semiconductors (Eindhoven, Netherlands). The microcontroller 440 can be programmed to perform signal processing (such as filtering), analysis (such as heart beat defect or arrhythmia detection) and provide the power management functions to maintain operation using minimal power. In accordance with some embodiments of the invention, the device 400 can include a wireless transceiver (e.g., Bluetooth, WiFi, and ZigBee) for transferring data to the sensor reading device 100 or a smart phone or similar device. The transceiver can be incorporated in the microcontroller 440 or provided as a separate component.

In accordance with some embodiments of the invention, a software latch can be used to keep the device 400 on once activated. In its packaged state, the device 400 can be disconnected from the battery by the switch 450, thus enabling the battery to remain nearly fully charged until it needs to be used. After unpacking the device, an NFC-enabled mobile device 200 can be brought into proximity of the antenna 412 of the device 400. The NFC interface 410 becomes energized and sends a “High” signal on NFC_Vout that enables (e.g. pulls high) a high-side latch of switch 452. The high-side latch of switch 452 closes the circuit that connects the battery 452 (and optionally, power from the interface 410, in the event that the battery is dead or not charged enough to boot the microcontroller) to microcontroller 440 and the other components of the device 400 and the battery now powers the system. The microcontroller 440 powers on and during or after initialization sets an output (e.g., the “Enable_Batt” pin) high. The “Enable_Batt” signal can be fed back to the switch 452 via an OR gate (e.g., constructed of diodes). This combination of hardware and software creates a power-on latch. Preferably, the mobile device 200 remains in proximity with the antenna 412 long enough for the microcontroller to set the “Enable_Batt” signal high and latch the switch closed. To power down the device, the microcontroller 440 sets the “Enable_Batt” signal low, causing the switch 452 to open disconnecting the battery 450. The battery 450 can be connected through the switch to the NFC interface 410 to transfer power to charge the battery. Alternatively, the battery 450 can be connected to a separate wireless or wired charging circuit, enabling the battery to be charged.

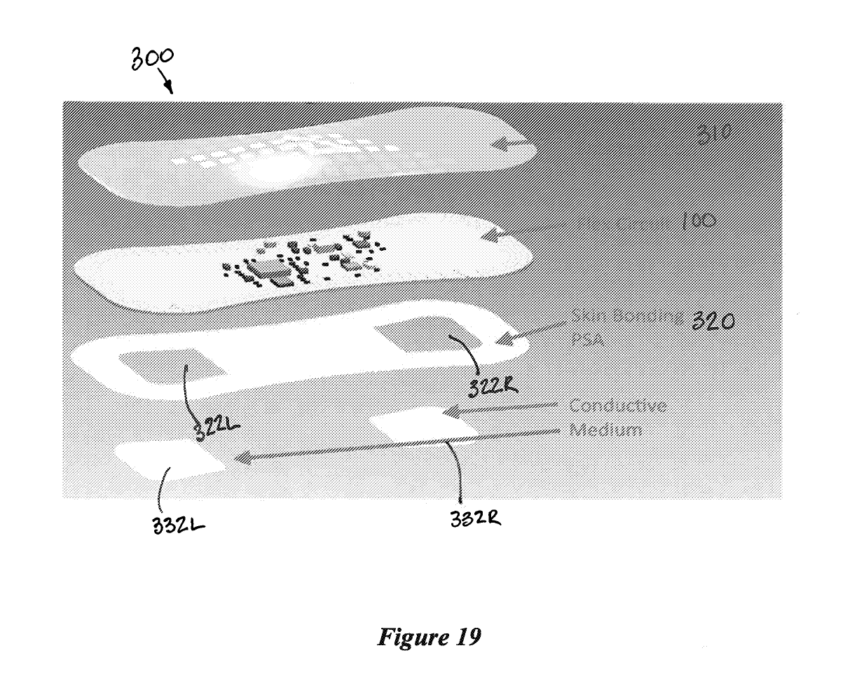

FIG. 19 shows a diagrammatic exploded view of the construction of a sensing device 300 or 400 according to the invention. The sensing device circuit 100 can be fabricated on a flexible printed circuit board (FPBC) 100A that includes the electrodes 132R, 132L, on the bottom surface of the FPCB 100A. The top of the FPCB 100A can be covered with an encapsulation layer 310, such as a flexible and/or stretchable waterproof layer (e.g., polyethylene, linear low-density polyethylene, polypropylene, polyimide, polyurethane, silicone, and other flexible polymer or copolymer materials in the range from 1 um to 300 um thick). The bottom of the FPCB 100A can be covered with a skin bonding pressure sensitive adhesive layer 320 (e.g., an acrylic pressure sensitive adhesive such as Flexcon (Spencer, Mass.) H-566, a silicon pressure sensitive adhesive, synthetic rubber based adhesives, a hydrocolloid base adhesive, a hydrogel based adhesive, in the range from 10 um to 125 um thick) enabling the device 300 to be adhered to the skin of a user. The pressure sensitive adhesive layer 320 can include cut-outs that align with the electrodes and enable the electrodes to directly contact the skin. Alternatively, the cut-outs can be covered with a conductive medium (e.g., a hydrogel material, such as Axelgaard (Fallbrook, Calif.) AG625 in the range from 0.010 in to 0.050 in thick) 332R, 332L, that provides immediate electrical conductivity between the skin and the electrodes 132R, 132L.

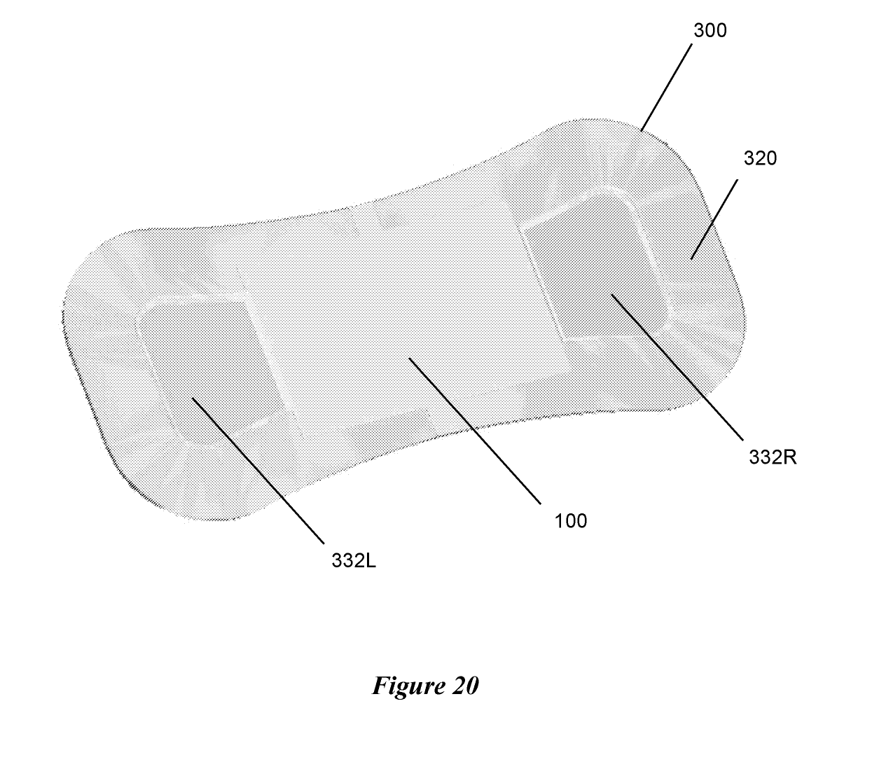

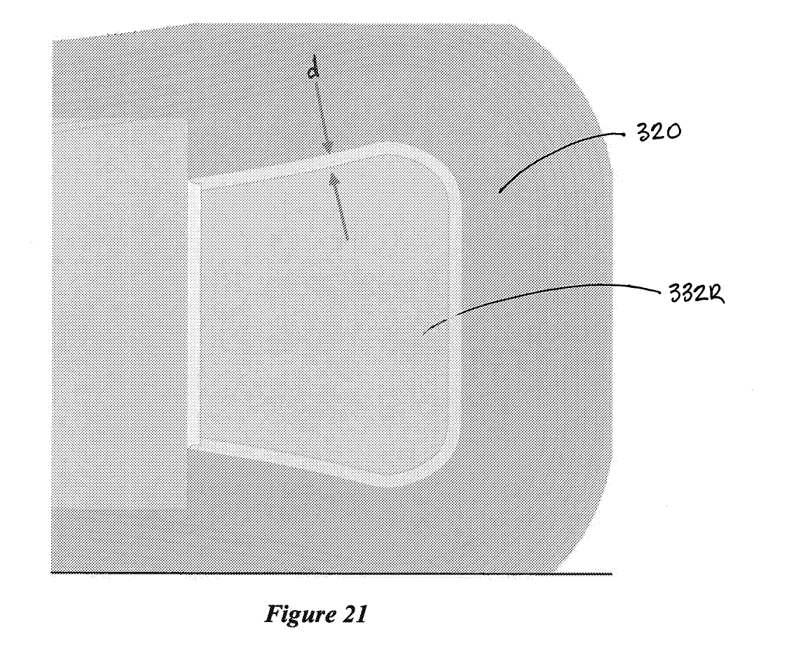

FIGS. 20 and 21 show a bottom view of the sensing device 300. The bottom surface can be covered by the pressure sensitive adhesive layer 320 (e.g., an acrylic pressure sensitive adhesive), with cut-outs or open spaces 322R, 322L, aligning with the electrodes 132R and 132L. In order to provide sealing to protect the FPCB 102 from the environment (e.g., water, sweat, etc.), the conductive medium can be larger in size than the cut-out, such that it overlaps the pressure sensitive adhesive layer 320 by a predefined distance, d. The overlap distance d can be in the range from 0.004 in to 0.020 in and preferably in the range 0.0075 to 0.0125 in when the hydrogel is approximately 0.025 in thick. The overlap distance d can vary depending on the thickness of the conductive medium and the skin bonding adhesive.

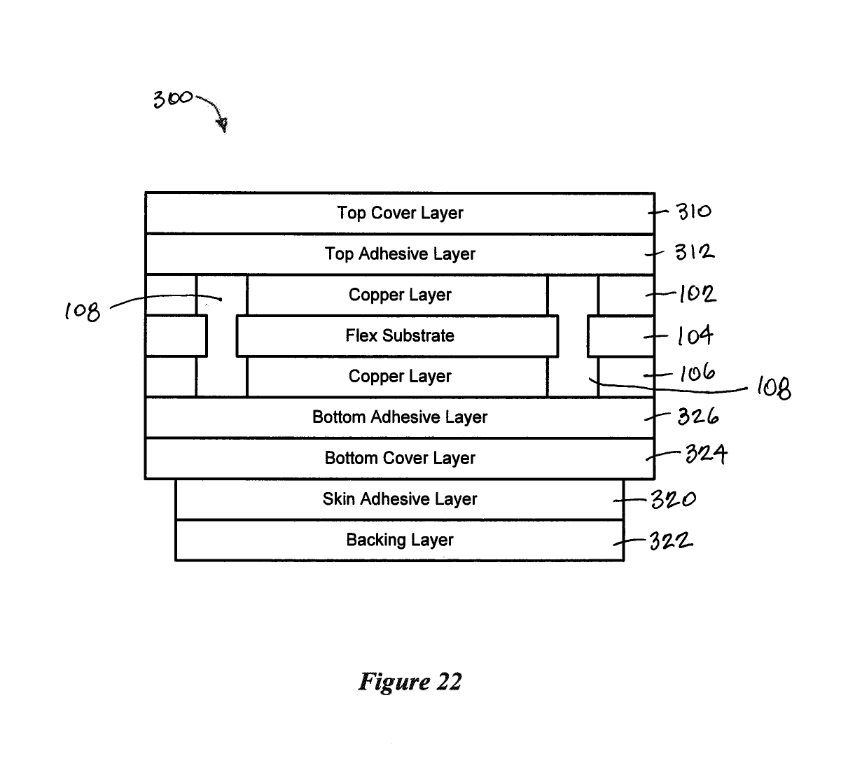

FIG. 22 shows a diagrammatic view of a stack-up of the materials that can be used to construct a sensing device 100, 300, 400, according to some embodiments of the invention. The FPCB 100A can include a flexible substrate (e.g., polyimide) 104 sandwiched between a first copper layer 102 and a second copper layer 106. The copper layers 102, 106 can be etched to form the circuit traces that connect the components that can be soldered to the flexible substrate 104. Conductive through holes 108 (such as plated or solder filled through holes) can be used to electrically connect features on the top surface of the FPCB 100A to the bottom surface of the FPCB 100A. The top cover layer 310 can be adhered by an adhesive (e.g., a pressure sensitive adhesive) 312 to the top of the first copper layer 102. The optional, bottom cover layer 324 can be adhered by an adhesive (e.g., a pressure sensitive adhesive) 326 to the top of the second copper layer 106. The skin bonding pressure sensitive adhesive layer 320 can be adhered to the bottom cover layer 324 or the second copper layer 106 and backing layer 322 can protect the skin bonding pressure sensitive adhesive layer 320 until it is ready to be attached to the skin of the user.

Other embodiments are within the scope and spirit of the invention. For example, due to the nature of software, functions described above can be implemented using software, hardware, firmware, hardwiring, or combinations of any of these. Features implementing functions may also be physically located at various positions, including being distributed such that portions of functions are implemented at different physical locations.

Further, while the description above refers to the invention, the description may include more than one invention.

What is claimed is:

1. A sensing device comprising:
a sensor tag integrated circuit operatively connected an antenna for receiving signals from a sensor reading device and producing an electrical current to power the sensor tag integrated circuit in response to the received signals; and
wherein the sensor tag integrated circuit is operatively connected to at least one sensor to receive biological signals of one or more biological conditions from the at least one sensor.

2. The sensing device according to claim 1 wherein the at least one sensor includes at least two electrodes.

3. The sensing device according to claim 2 further comprising a sensor signal processor connecting the at least two electrodes to the sensor tag integrated circuit.

4. The sensing device according to claim 2 further comprising an amplifier connecting the at least two electrodes to the sensor tag integrated circuit.

5. The sensing device according to claim 2 further comprising a sensor signal processor and an amplifier connecting the at least two electrodes to the sensor tag integrated circuit.

6. The sensing device according to claim 2 further comprising an analog to digital converter connecting the at least two electrodes to the sensor tag integrated circuit.

7. The sensing device according to claim 2 further comprising a programmable amplifier connecting the at least two electrodes to the sensor tag integrated circuit.

8. The sensing device according to claim 2 wherein the at least two electrodes sense bio-impedance of the skin.

9. The sensing device according to claim 2 wherein the at least two electrodes sense bio-potentials through the skin.

10. The sensing device according to claim 1 further comprising a microcontroller connecting the at least one sensor to the sensor tag integrated circuit.

11. The sensing device according to claim 10 further comprising a battery connected by a switch to the microcontroller wherein the switch includes a latch input that closes the switch connecting the battery to the microcontroller and wherein the sensor tag integrated circuit is connected to the latch input of switch such that when the sensor tag integrated circuit receives the signals from the sensor reading device, the sensor tag integrated circuit operates the switch to connect the battery to the microcontroller.

12. The sensing device according to claim 10 wherein the microcontroller is also connected to the latch input of the switch and after receiving power from battery, operates the switch to maintain the connection to the battery after the sensor tag integrated circuit stops receiving signals from the sensor reading device.

13. The sensing device according to claim 1 wherein the at least one sensor includes a temperature sensor.

14. The sensing device according to claim 1 wherein the at least one sensor includes an accelerometer or a gyroscope.

15. The sensing device according to claim 1 wherein the at least one sensor includes a light sensor.

16. The sensing device according to claim 1 wherein the at least one sensor senses bio-impedance.

17. The sensing device according to claim 1 wherein the at least one sensor senses bio-potential signals.

18. The sensing device according to claim 1 wherein the sensor tag IC and the antenna are mounted on flexible printed circuit board and flexible printed circuit board is encapsulated between a top cover layer and an adhesive layer.

19. The sensing device according to claim 18 further comprising a bottom cover layer between the flexible printed circuit board and the adhesive layer.

20. The sensing device according to claim 18 wherein the at least one sensor includes at least two electrodes mounted on a bottom surface of the flexible printed circuit board and the adhesive layer includes at least one cut-out that exposes at least one of the at least two electrodes through the adhesive layer.

21. The sensing device according to claim 20 further comprising at least one hydrogel layer covering the exposed electrode.

22. The sensing device according to claim 21 wherein the hydrogel layer overlaps at least a portion of the adhesive layer by at least 0.004 inches.
